(19) United States
(12) Patent Application Publication
(10) Pub. No.: US 2025/0361472 A1
(43) Pub. Date: Nov. 27, 2025

(54) Systems, Methods, and Compositions for Generation of Therapeutic Cells

(71) Applicant: National Resilience, Inc., San Diego, CA (US)

(72) Inventors: Richard M. Stoner, San Diego, CA (US); Dwight Baker, San Diego, CA (US); Brandon Rice, San Diego, CA (US)

(21) Appl. No.: 19/128,472

(22) PCT Filed: Nov. 10, 2023
(86) PCT No.: PCT/US2023/079368

§ 371 Date: May 8, 2025

Related U.S. Application Data
(60) Provisional application No. 63/383,233, filed on Nov. 10, 2022.

(51) Int. Cl. C12M 3/00; C12M 1/36; C12N 5/0781; C12N 5/0783; C12N 5/10; C12N 15/86; C40B 50/00; G01N 33/50; G01N 33/53; G01N 33/68

(52) U.S. Cl. CPC C12M 23/44; C12M 41/48; C12N 5/0635; C12N 5/0636; C12N 5/0646; C12N 5/10; C12N 15/86; C40B 50/00; G01N 33/5094; G01N 33/53; G01N 33/68; C12N 2510/00

(57) ABSTRACT

Systems, methods, and compositions can be used to generate a therapeutic cell. The system can be an fully closed, automated system. The system, methods and compositions can intake cells of a subject and modify the cells to generate therapeutic cells. Monitoring of the processes of the system and cells can be integrated into the system. Quality control metrics can be used to assess the quality of cells generated and assays to determine quality can be integrated and performed by the closed automated system.

### CROSS-REFERENCE TO RELATED APPLICATIONS

This application claims priority to U.S. Provisional Patent Application No. 63/383,233,filed on Nov. 10, 2022, the entire content of which is incorporated herein by reference in its entirety.

### BACKGROUND

Cell therapy can be used to treat diseases in individuals by providing cells to a subject. Autologous cell therapy can use cells removed from an individual, modified, and then reintroduced into the individual to provide a therapeutic effect. Cell therapies can be effective at treating cancer, hematologic condition, immune disorders, neurological disorders, and many other disorders, conditions, or diseases in a subject.

### SUMMARY

Recognized herein is a need for improved systems and methods for generating therapeutic cells with greater efficiency. The systems and methods described herein can be used for generating therapeutic cellular products with comprehensive quality control reports to consolidate assays and maximize use of the cellular products.

In an aspect, the present disclosure provides a system for generating therapeutic cells, wherein the system comprises: (a) a first module configured to receive a plurality of cells from a subject, wherein the first module is further configured to (i) select a subset of cells from the plurality of cells to yield selected cells; and (ii) introduce nucleic acids into the selected cells to yield modified cells; (b) a second module configured to culture the modified cells to generate expanded cells; and (c) a third module configured to harvest the expanded cells, thereby generating therapeutic cells, wherein the third module is further configured to (i) generate a formulation comprising the therapeutic cells, and (ii) preserve the therapeutic cells; wherein the system is a closed, automated system.

In some embodiments, the first module is further configured to activate the selected cells to generate activated cells.

In some embodiments, the first module is configured to introduce nucleic acids into the selected cells by transduction to generate transduced cells. In some embodiments, the first module is configured to introduce nucleic acids into the selected cells by transfection.

In some embodiments, the system is further configured to identify at least one characteristic of the plurality of cells, the selected cells, the activated cells, the transduced cells, or the expanded cells.

In some embodiments, the system further comprises a fourth module configured to monitor the first module or the second module.

In some embodiments, the fourth module is further configured to identify a characteristic of the plurality of cells, the selected cells, the activated cells, the transduced cells, or the expanded cells. In some embodiments, the system is further configured to perform a quality analysis on the expanded cells to determine a quality of expanded cells.

In some embodiments, the quality analysis comprises forming a nucleic acid library, optionally wherein the nucleic acid library comprises a next generation sequencing library. In some embodiments, the quality analysis comprises identifying a vector copy number (VCN), a replication competent lentivirus (RCL), a chimeric antigen receptor (CAR) expression level, an immunophenotyping, or a combination thereof.

In some embodiments, the at least one characteristic comprises a phenotype of a cell of the plurality of cells, the selected cells, the activated cells, the transduced cells, or the expanded cells. In some embodiments, the phenotype of the cell is a T cell, a B cell, or a NK cell.

In some embodiments, the at least one characteristic comprises a genotype of a cell of the plurality of cells, the selected cells, the activated cells, the transduced cells, or the expanded cells.

In some embodiments, the at least one characteristic comprises a viability of a cell of the plurality of cells, the selected cells, the activated cells, the transduced cells, or the expanded cells.

In some embodiments, the at least one characteristic comprises a presence of a contaminant. In some embodiments, the contaminant is a bacterial contaminant, a mold contaminant, a viral contaminant, a yeast contaminant, or a mycoplasma contaminant. In some embodiments, the contaminant is an endotoxin.

In some embodiments, the at least one characteristic comprises an image of a cell of the plurality of cells, the selected cells, the activated cells, the transduced cells, or the expanded cells.

In some embodiments, the at least one characteristic comprises a potency of a cell of the plurality of cells, the selected cells, the activated cells, the transduced cells, or the expanded cells.

In some embodiments, the at least one characteristic comprises a pH, a temperature, an oxygen level, or a metabolic composition of a cell of the plurality of cells, the selected cells, the activated cells, the transduced cells, or the expanded cells.

In some embodiments, the first module is further configured to monitor the selected cells or modified cells. In some embodiments, the second module is further configured to monitor the expanded cells. In some embodiments, the plurality of cells are in a blood bag or apheresis bag. In some embodiments, the plurality of cells are from a biological sample. In some embodiments, the plurality of cells comprise immune cells.

In some embodiments, the third module is further configured to cryopreserve the therapeutic cells. In some embodiments, the therapeutic cells are chimeric antigen receptor (CAR) T cells.

In another aspect, the present disclosure provides a method for generating therapeutic cells, wherein the method comprises: (a) receiving a plurality of cells from a subject; (b) selecting a subset of cells from the plurality of cells to yield selected cells; (c) introducing nucleic acids into the selected cells to yield a modified cell; (d) culturing the modified cell to generate expanded cells; (e) harvesting the expanded cells, thereby generating therapeutic cells; (f) generating a formulation comprising the therapeutic cells; and (g) preserving the therapeutic cells, wherein the method is performed in a closed automated system.

In some embodiments, the method further comprises prior to c) and subsequent to b), activating a cell from the selected cells to yield an activated cell, wherein c) comprises introducing nucleic acids into the activated cells.

In some embodiments, the introducing nucleic acids into the selected cells comprises transfection. In some embodiments, the introducing nucleic acids into the selected cells comprises transduction to generate transduced cells.

In some embodiments, the transduction comprises using a viral vector. In some embodiments, the viral vector is a lentiviral vector.

In some embodiments, the method further comprises identifying at least one characteristic of the plurality of cells, the selected cells, the activated cells, the transduced cells, or the expanded cells.

In some embodiments, the method further comprises performing quality analysis on the expanded cells to determine quality of expanded cells.

In some embodiments, the quality analysis comprises forming a nucleic acid library, optionally wherein the nucleic acid library comprises a next generation sequencing library. In some embodiments, the quality analysis comprises identifying a vector copy number (VCN), a replication competent lentivirus (RCL), a chimeric antigen receptor (CAR) expression level, an immunophenotyping, or a combination thereof.

In some embodiments, the at least one characteristic comprises a phenotype of a cell of the plurality of cells, the selected cells, the activated cells, the transduced cells, or the expanded cells. In some embodiments, the phenotype of the cell is a T cell, a B cell, or a NK cell.

In some embodiments, the at least one characteristic comprises a genotype of a cell of the plurality of cells, the selected cells, the activated cells, the transduced cells, or the expanded cells.

In some embodiments, the at least one characteristic comprises a viability of a cell of the plurality of cells, the selected cell, the activated cell, the transduced cell, or the expanded cells.

In some embodiments, the at least one characteristic comprises a presence of a contaminant. In some embodiments, the contaminant is a bacterial contaminant, a mold contaminant, a viral contaminant, a yeast contaminant, or a mycoplasma contaminant. In some embodiments, the contaminant is an endotoxin.

In some embodiments, the at least one characteristic comprises an image a cell of the plurality of cells, the selected cell, the activated cell, the transduced cell, or the expanded cells. In some embodiments, the plurality of cells are in a blood bag or apheresis bag. In some embodiments, the plurality of cells are from a biological sample. In some embodiments, the plurality of cells comprise immune cells.

In some embodiments, the preserving comprises cryopreserving the therapeutic cells.

In some embodiments, generating a formulation comprises adding a cryoprotectant to the therapeutic cells.

In another aspect, the present disclosure provides a non-transitory computer readable medium comprising instructions that, when executed by a computer processor, cause the computer processor to automatically control a closed system to (a) receive a plurality of cells from a subject; (b) select a subset of cells from the plurality of cells to yield selected cells; (c) introducing nucleic acids into the selected cells to yield modified cells; (d) culture the modified cells to generate expanded cells; (e) harvest the expanded cells, thereby generating therapeutic cells; (f) generate a formulation comprising the therapeutic cells; and (g) preserve the therapeutic cells.

In another aspect, the present disclosure provides a non-transitory computer readable medium comprising instructions that, when executed by a computer processor, cause the computer processor to automatically control a closed system to perform the method described herein.

In another aspect, the present disclosure provides a non-transitory computer readable medium comprising instructions that, when executed by a computer processor, cause the computer processor to automatically control the system described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and the disclosure will be obtained by reference to the following detailed description that sets forth illustrative embodiments, in which the principles of the invention are utilized, and the accompanying drawings (also “Figure” and “FIG.” herein), of which:

FIG. 1 shows a schematic of an example system for generating therapeutic cells.

FIG. 2 shows a computer control system that is programmed or otherwise configured to implement methods provided herein.

FIG. 3 shows a schematic of an example system for generating therapeutic cells.

### DETAILED DESCRIPTION

Provided herein are systems, methods, and compositions for generating therapeutic cells. The cells generated using the systems, methods, and compositions can be used to treat or ameliorate a disorder or disease in a subject. The systems, methods, and compositions can increase access for subjects to cell therapies, such as autologous cells therapy. New autologous cell therapies can be manufactured via a distributed, self-contained cell therapy manufacturing platform such as those disclosed herein. Methods of generating autologous cell therapies can have prohibitively high manufacturing costs, and can also have weeks or months-long vein-to-vein turnaround times. Without the systems, methods, and compositions disclosed herein, a patient may be unable to have access to the autologous cell therapies, due to the high costs and long turn over time.

Autologous cell therapies can provide significant therapeutic benefits to a subject in need. Autologous cells can be derived from the subject by extraction of a fluid or tissues from a subject. The cells can then modified ex vivo to produce a phenotype on the cells that can produce a therapeutic effect. The modified cells can then be provided back to the subject, in which a therapeutic benefit can be conferred to the subject. These autologous cell therapies can comprise advantages over other cell based therapies as the cells are derived from the end recipient of the treatment. Non-autologous cell therapies can result in immune complications where the transplanted cells can cause damage to the subject's cells, or the transplanted cells can be rejected by the subject. For example, graft versus host disease (GVHD) can be caused where the transplanted immune cells attack the subject cells resulting in inflammation or cell death. This can result in the subject having chronic and potentially life threatening side effects from the cell therapy. Additionally, to combat potentially immune responses to the therapies, the subject may need to be placed on immunosuppressants which can lower the immune response of the subject and result in higher disease susceptibility. Autologous therapies, because the cells are derived from the subject that is being treated, do not suffer from these types of side effects as the immune system of the subject will not generally respond adversely to cells that were originally derived from the same person.

However, because autologous cell therapies use cells from a subject that is to be treated, the therapies are personalized for each individual and can require significant time and cost to produce the cells of interest, and cells cannot generally be mass manufactured as would be possible for other forms of treatment such as antibodies or small molecule drugs. As such, there is a need for an efficient, cost effective, and accurate system for generating autologous cells for therapy. The systems disclosed herein can provide a fully automated platform that takes a cell sample from a subject (such as a blood bag or apheresis bag) as input and provide cell isolation, washing, gene integration, activation, expansion, QC testing, final fill, and finish of the product for therapy and product archival. This platform solution can thus decrease the overall number of steps in a manufacturing process and enable inline and integrated release test strategies to improve drug quality while reducing the manufacturing cost of goods.

The system can allow for fully-distributed operation at or near the point of care, and can significantly reduce the costs and time associated with freezing, sending, receiving, and thawing modified cells from a centralized manufacturing facility. The system can comprise advantages by integrating the manufacturing processes, from apheresis to released drug products, with inline monitoring, quality checks, and tracible auditable data collection with reporting throughout the workflow of the system. This integration of manufacturing and monitoring can allow the for the product and process to be monitored at all stages in the workflow to identify any issues in the process. By integrating assays of cell quality and identity, the systems can shave weeks off turnaround times and eliminate logistics challenges with sending samples out for testing.

Consolidation of the assays of the system and methods described herein can allow for minimizing the amount of the sample utilized for an assay. Minimizing the amount of a sample utilized for the assay can decrease the total time for an assay or increase the efficiency of the system compared to that of other systems and methods used for generating therapeutic cells. The consolidated workflow of the system and methods described herein can provide for production of therapeutic cells (e.g., CAR T cells) as well as output reports relating to the quality of the system (e.g., a progress report) and the quality of the final expanded cells (e.g., release report). This organization can allow for a more streamlined approach to generating populations of therapeutic cells.

Next generation sequencing data and RNA readouts can be combined with bioinformatics in a data analysis pipeline to provide a comprehensive overview of the final cell product.

The system and methods provided herein can use a sample from a human subject (e.g., a human patient suffering from a disease, disorder, or condition).

A further advantage of the systems, methods, and compositions provided herein can be to track patients and detect potential contaminants through sequencing reads.

The systems, methods, compositions, can be flexible and accommodating a diversity of different cell therapy modalities. For example, the system can be able to accommodate a diversity of different immune cell types (T cells, NK cells, B cells), targets (CD19, BCMA, etc.), as well as different modification strategies (viral/non-viral, single gene/multi-gene).

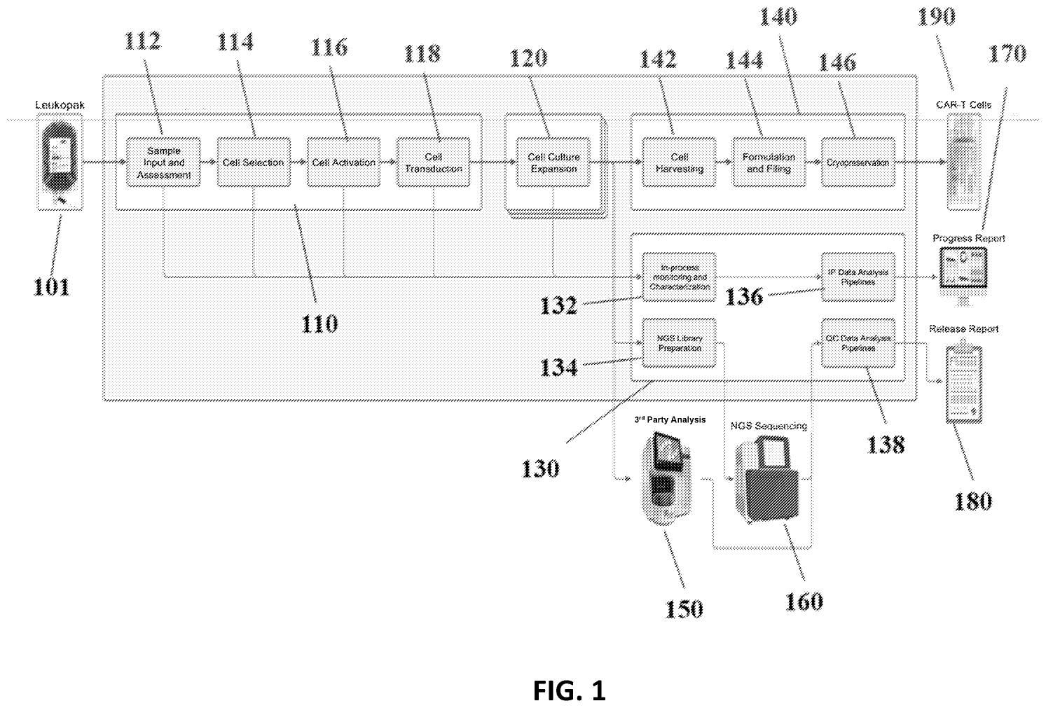

An example system of the disclosure is shown in FIG. 1. A subject can be suffering from a disorder and can benefit from treatment with cell therapy. A sample can be taken from a subject and collected in a blood bag or apheresis bag. A blood bag 101 comprising cells of a subject can be hooked up a system. The cells can be moved into module 110 which performed initial reactions on the cells. The module 110 can receive the sample via sample input and assessment 112 which can receive samples of whole blood or apheresis products. The module can provide a fast early assessment of the cell count and viability along with a immunophenotyping of the starting material to provide information on the starting material for later processes. After sample input and assessment, the module 110 can perform a cell selection 114 to select for a set of cells, for example, naive T-cells, for modification and activation. Cell activation 116 can then be performed by introducing a stimuli to the cells (e.g., T-cells) to generate activated cells. Cell activation 116 can be performed by introducing beads or another product with antibodies that interact with the cells causing activation. Cell transduction 118 can then performed by adding in nucleic acids and a nucleic acids vector to integration of nucleic acids. Cells transfection of other forms of the nucleic acid manipulation can also be performed. Cell transduction 118 can include introduction of exogenous genes into the cells, for example, chimeric antigen receptors (CARs) and can generate CAR-T cells. The newly transduced and activated cells can then be moved to module 120 to allow for cell culture expansion and proliferate the cells. Module 130 can be configured to perform monitoring of the process of module 110 and 120. Module 130 can be directly integrated with module 110 and 120 or can be a standalone module. Module 130 can perform in-line monitoring and characterization 132 and characterize cells in other modules. For example, the phenotype of cells can be characterized. Module 130 can also perform NGS library preparation 134 by extracting nucleic acids from the cells and subjecting these nucleic acids to library preparation, single cell barcoding, and NGS sequencing 160. The expanded cells can also be subjected to third party analysis 150 to determine the quality of these expanded therapeutic cells by a sample output that remains sterile. Module 130 can use the data from monitoring, sequencing, and third part analysis to an inputted into an in-line processing data analysis pipeline 136 and a Quality Control Data analysis pipeline 138. The pipelines 136 and 138 can generate reports such as a progress report 170 relating to the status of the systems and cells, and a release report 180 relating to the quality metrics of the final expanded cells.

Upon expansion of cells, the cells are moved to module 140 for harvesting 142, formulation and filling 144, and cryopreservation 146. The cells can be harvested and then aliquoted in a formulation and the preserved to longer storage. The cells can be a ready for use by a subject such as therapeutically ready cells 190, such as CAR-T cells. The cells can then be provided to a subject for treatment.

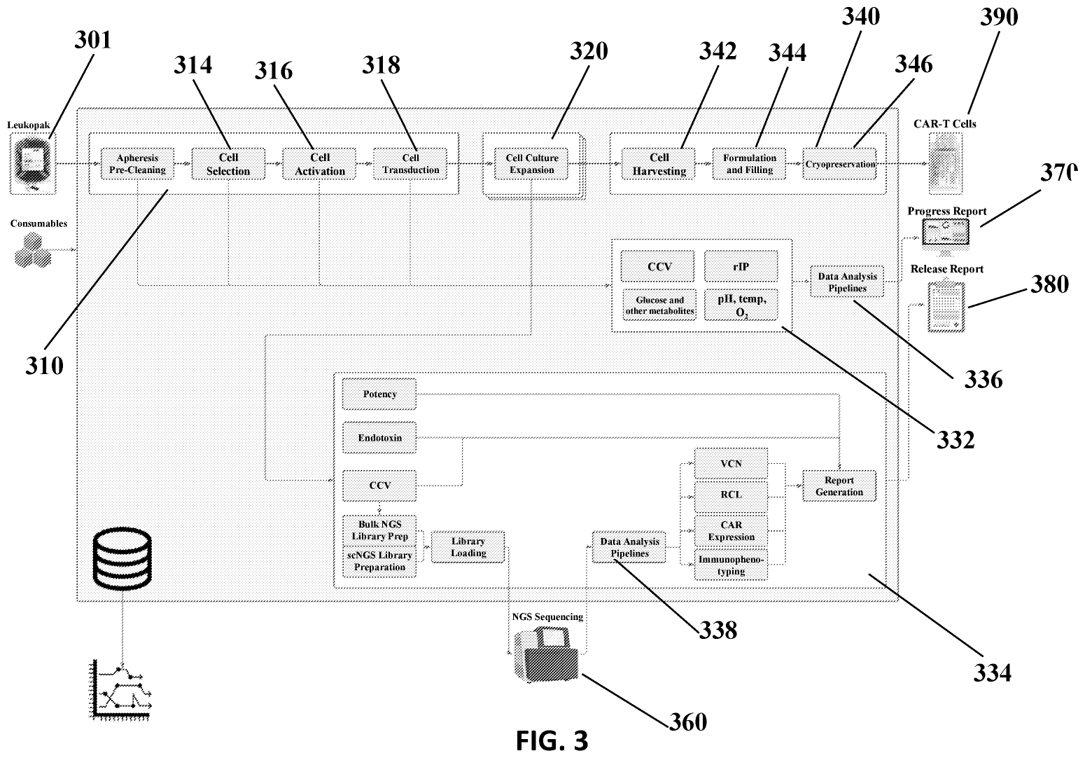

Another example system of the disclosure is shown in FIG. 3. A subject may be suffering from a disorder and can benefit from treatment with cell therapy. For example, the subject can be suffering from infectious disease, autoimmune disease, or cancer. A sample can be taken from a subject and collected in a blood bag or apheresis bag. A blood bag 301 comprising cells of a subject can be connected to a system. A blood bag 301 from a subject can undergo apheresis pre-cleaning. Apheresis can comprise removal of blood plasma by the withdrawal of blood and separation into plasma and cells. The cells can be moved into module 310 which can perform initial reactions on the cells. The module 310 can receive the sample via sample input and assessment which can receive samples of whole blood or apheresis products (e.g., plasma and cells). The module 310 can provide a fast early assessment of the cell count and viability along with a immunophenotyping of the starting material to provide information on the starting material for later processes. After sample input and assessment, the module 310 can perform a cell selection 314 to select a set of cells. Cells can be, for example, naive T-cells, B-cells, NK cells, macrophages, dendritic cells, or other immune cells. Cell selection 314 can produce a population of selected cells from the plurality of cells of the sample. Selected cells may then be used for modification and activation. Cell activation 316 can then be performed by introducing a stimuli to the cells (e.g., T-cells) to generate activated cells. Cell activation 316 can be performed, for example, by introducing beads or another product with antibodies that interact with the cells causing activation. Cell activation 316 can be performed by introducing an antigen-presenting cell or cytokine. Cell activation 316 can produce a population of activated cells from the selected cells. Cell transduction 318 can then performed by adding nucleic acids and a nucleic acid vector to integration of nucleic acids. The vector can be, for example, a viral vector. The viral vector can be, for example, a lentiviral vector, an adenoviral vector, or an adeno-associated viral vector, or a retroviral vector. Cells transfection of other forms of the nucleic acid manipulation can also be performed. Cell transduction 318 can include introduction of exogenous genes into the cells, for example, chimeric antigen receptors (CARs) and can generate CAR-T cells. Cell transduction 318 can produce a population of transduced cells from the activated cells. The transduced and activated cells can then be moved to module 320 to allow for cell culture expansion and proliferate the cells. A quality control module can be configured to perform monitoring of the process of module 310 and 320. The quality control module can comprise an In-Process Monitoring and Characterization Module 332 and a release quality control (QC) and characterization module 334. The In-Process Monitoring and Characterization Module 332 and release quality control (QC) and characterization module 334 can directly integrate with module 310 and 320 or each component can be a standalone module. The In-Process Monitoring and Characterization Module 332 and release quality control (QC) and characterization module 334 can characterize cells in other modules. For example, the phenotype of selected cells, activated cells, transduced cells, and/or expanded cells can be characterized. The genotype can be characterized of selected cells, activated cells, transduced cells, and/or expanded cells. The In-Process Monitoring and Characterization Module 332 can characterize the metabolites of selected cells, activated cells, transduced cells, and/or expanded cells. The In-Process Monitoring and Characterization Module 332 can characterize the pH, the temperature, and/or the oxygen level of selected cells, activated cells, transduced cells, and/or expanded cells. The In-Process Monitoring and Characterization Module 332 can characterize a cell count and viability of selected cells, activated cells, transduced cells, and/or expanded cells. Measurements of the In-Process Monitoring and Characterization Module 332 can be collected together or separately. The release quality control (QC) and characterization module 334 can also perform next generation sequencing (NGS) library preparation by extracting nucleic acids from the cells and subjecting these nucleic acids to library preparation, single cell barcoding, and NGS sequencing 360. The expanded cells can also be subjected to third party analysis to determine the quality of these expanded therapeutic cells by a sample output that remains sterile. The In-Process Monitoring and Characterization Module 332 can use the data from monitoring, sequencing, and third party analysis to an inputted into an in-line processing data analysis pipeline 336. The release quality control (QC) and characterization module 334 can use the data from monitoring, sequencing, and third party analysis to an inputted into a Quality Control Data analysis pipeline 338. The Quality Control Data analysis pipeline 338 can evaluate vector copy number (VCN), replication competent lentivirus (RCL), expression level of chimeric antigen receptor (CAR), and/or immunophenotyping of cells. The pipelines 336 and 338 can generate reports such as a progress report 370 relating to the status of the systems and cells, and a release report 380 relating to the quality metrics of the final expanded cells. The reports can be generated together or separately.

Upon expansion of cells, the expanded cells can be moved to module 340 for harvesting 342, formulation and filling 344, and/or cryopreservation 346. The cells can be harvested and then aliquoted in a formulation and the preserved to longer storage. The cells can be a ready for use by a subject such as therapeutically ready cells 390, such as CAR-T cells. The cell products (e.g., CAR-T cells) can then be provided to a subject for treatment. Following cryopreservation, the cell products (e.g., CAR-T cells) can be stored for at least about, at most about, or about 1 day, 2 days, 3 days, 4 days, 5 days, 6 days, 1 week, 2 weeks, 3 weeks, 1 month, 2 months, 3 months, 4 months, 6 months, 8 months, 1 year, 2 years, 3 years, 4 years, 5 years, or a range between any of these two values.

In various aspects, a sample can be collected from a subject. A sample can be a sample 101, 301. The subject can be a human subject. The subject can be an individual that is suffering from a disease, disorder, or condition. For example, the subject can have cancer. The subject can have an autoimmune disorder. The subject can have an infectious disease. The sample can be a blood sample, plasma sample, serum sample, bone marrow sample a urine sample, a cerebrospinal fluid sample, a pleural fluid sample, a rectal sample, a vaginal sample, a stool sample, a sputum sample, and/or a lymph sample. The sample can be a solid sample. The sample can be a liquid sample. The sample can be subjected to separation such to isolate a type of cells, for example, a leukocyte or erythrocyte. The sample can comprise immune cells. For example, the sample can comprise lymphocyte, T-cells, NK cells, B cells, leukocytes, macrophages, dendritic cells, monocytes, mast cells neutrophils or other immune cells. The sample can comprise naïve T-cells or B-cells. The sample can comprise mature T-cells or B-cells. The sample can comprise stem cells.

In various aspects, the cells from a sample can be selected for and isolated. Cell selection and isolation can be performed in module 110, 310 as corresponding to cell selection 114, 314. Cell selection can comprise mechanical separation such as centrifugation. For example, a sample can be subjected to centrifugation and certain cells can remain in a supernatant fraction and other cells can be present in the pellet fraction. The sample can be subject to coagulation or precipitation, such that a subset of cells is removed from a solution. Cells can be selected based on an expression or presence of a biomarker. For example, cell can express a membrane protein or extracellular protein that is accessible on an outside surface of a cell. For example, a cell can express CD19, BCMA, or other biomarker associated with a cell of interest. A binding agent can be specific for a particular membrane protein or extracellular protein and can bind to cells that express the particular membrane or extracellular protein. A binding agent, such an antibody can be conjugated to a support which can be isolated. The support can be a bead. The support can be a magnetic bead. The sample can be subjected to a magnetic field such to isolate the bead (and bound cells) from the rest of the sample. Cells can be subject to multiple rounds of selection. For example, a plurality of cells can be first subjected a first selection based on size and then another selection based on binding to a particular analyze. For example, a plurality of cells can be first subjected a first selection based on the presence of a first phenotypic or genotypic feature and then another selection based on the presence of a second phenotypic or genotypic feature.

Testing aliquots can be taken directly from a bioreactor, frozen samples, or in-process development and introduced into the module 130. Sample inputs can be aliquoted out into a format that can be used within the module 130 that utilizes multi-omics data from sequencing and computer vision analysis to be inputted into an off-line processing data analysis pipeline 136, 336 and a Quality Control Data analysis pipeline 138, 338. The analysis pipelines can be executed in parallel and can leverage some information from intermediate steps that generate reports such as a progress report 170, 370 relating to the status of the systems and cells and a release report 180, 380 relating to the quality metrics of the final expanded cells. The analysis pipelines can be executed separately. Each assay can be provided from the analysis as a single result. Each assay can be combined with other assays to deliver a multi-omics interpretation of the cells and product. A module, (e.g., a cell count and viability (CCV) module), can be utilized within the QC device, bioreactor, or as a single module or instrument.

In various aspects, the cells can be activated to generate a phenotype. Activation can be performed using module 110, 310 corresponding to cell activation 116, 316. For example, the cells can be T-cell or B-cells and can be activated to express a T-cell or B-cell subtype. The cell can be activated by another cell. For example, an antigen presenting cell (APC) (e.g., a dendritic cell) can be allowed to contact a T-cell and generate a cellular response. The APC can comprise multiple signaling molecule and can provide multiple different stimuli to activate the cells. The cells can be activated by a soluble molecule. For example, a solution of soluble polypeptide can be introduced and the cells can be allowed to contact the soluble polypeptide. For example, the soluble polypeptide can comprise a growth factors, cytokines, interleukins, or other signaling molecule. The cells can be activated by an antibody. An antibody can be conjugated to a support.

In various aspects, the nucleic acids can be introduced into the cells. The introduction of nucleic acids can be performed using module 110, 310. Forms of nucleic acid instruction can correspond to cell transduction 118, 318. The nucleic acids can be RNA or DNA. The cells can be transduced, transfected, or otherwise provide nucleic acids for integration into the genome of the cell. For example, a chimeric antigen receptor (CAR) can be encoded in a viral vector. The viral vector can be allowed to insert genetic material into a cell and incorporate nucleic acids from the viral vector into the cell genome. The cells can be transfected using mRNA transfection. For example, the cells can be subjected to a transfection agent and an mRNA to allow uptake of the mRNA into cells. The introduction of the nucleic acids into the cells can allow for the expression of exogenous polypeptides. For example, the nucleic acids can encode for a CAR can allow for the expression of the CAR on a T-cell to generate a CAR-T cell.

In various aspects, the cells can be expanded to generate additional cells. The introduction of nucleic acids can be performed using module 120, 320. The expansion of cells can comprise proliferating a cell of interest and generating a larger quantity of cells comprises a phenotype of interest. The expansion can comprise culturing a cell in a media. The cell can be passaged into fresh media and allowed to proliferate and generate additional cells with the same genotype or phenotype. The expansion can comprise subject the cells to a continuously flowing media.

In various aspects, the cells can be harvested. Cell harvesting can be performed using module 140, 340 corresponding to cell harvesting 142, 342. The harvested cells can be cells that have been subjected to modification, activation, or expansion. Harvesting of cell can comprise moving cells from a first container to a new container. The harvesting of cells can comprise preserving the cells. For example, the cells can be cryopreserved. The harvesting of cells can comprises introduction of reagents to generate cell formulations. The cells can then be combined with reagent to generate formulations. Formulation generation can be performed in module 140, 340 corresponding to formulation and filling 144, 344. The formulations can allow the cells to have improved stability, or viability, increased shelf life, improved therapeutic efficacy, or other parameters. The formulations can comprise cryoprotectants. Harvesting can comprise aliquoting cells to a new sterile container for distribution to an individual. The cells can then be cryopreserved. Cryopreservation can be performed by module 140, 340 corresponding to cryopreservation 146, 346.

The system can comprise a first module that is configured to receive a plurality of cells from a subject, such as module 110, 310. The plurality of cells can be immune cells or blood cells from the subject. The system can be configured to allow a sample collection apparatus to interface with the system. For example, a blood bag or apheresis bag can be provided to the system and a tubing or other interface can allow for the sample in the blood bag or apheresis bag to enter the system.

The system can comprise a module that selects a subset of cells from the plurality of cells to yield selected cells, such as module 110, 310. Selection of cells can be performed as described elsewhere herein, or using other methods known in the art. The system can comprise a module to introduce nucleic acids into the selected cells to yield a modified cell. Introduction of nucleic acids can be performed as described elsewhere herein, such as using transduction or transfection.

The system can comprise a module configured to culture the cells to generate expanded cells, such as module 120, 320. The cells can be modified via genetic engineering or can be activated via introduction of stimuli to a cell. The cells (e.g., activated or modified cells) can exhibit a genomic or phenotypic characteristic, and the genomic or phenotypic characteristic can comprise a therapeutic effect upon introduction to a subject. Production and proliferation of these cells can be performed to generate a larger quantity of cells. Expansion of cells can be performed as described elsewhere herein, such as via culturing of cells in fresh media.

The system can comprise a module configured to harvest cells, such as module 140, 340. The harvested cells can be cells that have been subjected to modification, activation, expansion, or other activities performed in other modules of the system. The harvesting of cells can be performed as described elsewhere herein. The harvesting of cells can comprise preserving the cells. For example, the cells can be cryopreserved. Harvesting can comprise aliquoting cells to a new sterile container for distribution to an individual. The harvesting can be automated such that a human operator cannot contact the cells while in system and reduce potential external contamination.

The system can comprise modules that monitor reactions and processes of the system and characterize cells, such as modules 130, 150, 350, 160, and 360. A given module of the system can be integrated monitoring along with other processes. For example module can be configured to select for cells and can also monitor the process and accuracy of the selection. Monitoring can be also performed by a separate module that monitors the processes of other modules.

Monitoring can comprise performing assays on the cells that are inputted, outputted or actively processed in a module. Monitoring can comprise visual monitoring of cells. Monitoring can comprise determination of chemical compounds secreted or expressed by a cell. Monitoring can comprise determination of polypeptides expressed by a cell. Monitoring can comprise determination of polynucleotides expressed by a cell. Monitoring can comprise determination of a genomic sequence of a cell.

In various aspects, a sequence of a nucleic acid in a cell is determined. The determination of the sequencing can comprise a sequencing technique. The sequencing technique can be a next generation sequencing technique. The sequencing technique can comprise library preparation. The library preparation can be performed in module 130 corresponding to NGS library preparation 134. The library preparation can be performed in module 334, which can comprise bulk NGS library preparation and/or single-cell NGS library preparation. For example, the next generation sequencing can comprise whole genome sequencing, exome sequencing, targeted sequencing, RNA sequencing, methylation sequencing or combinations thereof. The next generation sequencing can comprise any sequencing method and can be a sequencing-by-synthesis, sequencing-by-ligation, pyrosequencing, Sanger sequencing, ion torrent sequencing nanopore sequencing, or single molecule sequencing. The NGS sequencing can be performed in module 130, 334 and can use a next generation sequencer 160, 360. The next generation sequencing can be single-cell next generation sequencing.

The sequencing method can comprise a next generation sequencing or a non-next generation sequencing assay. For example, the sequencing method can comprise polony sequencing, high throughput pyrosequencing, bead array sequencing, ion semiconductor sequencing, RNAP sequencing, nanopore DNA sequencing, DNA nanoball sequencing, or massively parallel signature sequencing.

Sequencing can provide a quality analysis of the cells of the system described herein. The quality analysis can comprise identifying a vector copy number (VCN), a replication competent lentivirus (RCL), a chimeric antigen receptor (CAR) expression level, an immunophenotyping, or a combination thereof. The vector copy number can refer to the number of vector genomes integrated into the genome of a target cell. The quality analysis can comprise monitoring of CAR potency and specificity.

Monitoring can comprise the detection of metabolites, polypeptides, or other macromolecules. Monitoring of metabolites, polypeptides or other macromolecules can comprise conducting affinity or binding based assays to detect the presence of given metabolite, polypeptide, or other macromolecule. For example, an assay can comprise the use of antibody specific to a metabolite, wherein the antibody can be tagged or otherwise identified via a signal. Assays can comprise detection of fluorescent signal, luminescent signal, colorimetric signal, radioactive signal or other detectable signal. Monitoring can comprise the use of chromatography or other separation technique. For example, solution can be added to a column to allow metabolites in a solution to be separated via a physical property and detected. The metabolites, polypeptides, or other macromolecules can be present in a solution and may have been excreted by a cell into a media, The metabolites, polypeptides, or other macromolecules can be present on the surface of a cell. For example, a surface or membrane protein can be present or exposed on a surface of a cells. Detection of the surface or membrane protein can be performed and indicate that the cell is a particular phenotype. The monitoring can allow for the selection of cells of interest. The monitoring can allow for the quality of a cell to be assessed. Upon assessment of the characteristics of a cell, the cell can be allowed to be activated, modified, expanded, or other reactions can be performed on a cell as described elsewhere herein.

Monitoring can comprise visual detection or imaging of a cell. The modules of the system can integrate a camera, microscope, or other visual implement for visualizing a cell or cell environment. Visual imaging of the cell can allow for the detection of a viability of a cell or allow for a determination of an overall health of a cell. Visual detection can also allow for module to assessed for proper function. Contamination and sterility can also be determined by visual detection in the system modules.

Characterization of the cells can be performed to quality control and check for the efficacy of the resulting cells. Characterization can use assays as described elsewhere, for example, viability assays, assays for detection of a genotype or phenotype. Characterization assays can use small sample volumes to maximize the yield of cells. In line monitoring can allow for the generation of more efficacious cells, as a given step can be monitored and the product of the step can be subjected to quality metric before moving a cell to a subsequent step. Characterization can be performed in module 130 and can correspond to In-Process Monitoring and Characterization 132, 332. Characterization can be performed in module 150 and can be sourced to an analysis system outside of the closed automated system.

In-Process Monitoring and Characterization 132, 332 can comprise analysis of cell count viability (CCV), pH, temperature, and/or oxygen levels. The amount of metabolites (e.g., glucose) can also be measured. Receptor interacting protein (rIP) kinases can play roles in cellular signaling during infection and inflammation. In-Process Monitoring and Characterization 132, 332 can also comprise measuring rIP levels of cells. A pH of a cell generated by the systems or methods described herein can be at least about 5.0, at least about 5.5, at least about 6.0, at least about 6.1, at least about 6.2, at least about 6.3, at least about 6.4, at least about 6.5, at least about 6.6, at least about 6.7, at least about 6.8, at least about 6.9, at least about 7.0, at least about 7.1, at least about 7.2, at least about 7.3, at least about 7.4, at least about 7.5, at least about 8.0, or greater than about 8.0. A pH of a cell generated by the systems or methods described herein can be at most about 8.0, at most about 7.5, at most about 7.4, at most about 7.3, at most about 7.2, at most about 7.1, at most about 7.0, at most about 6.9, at most about 6.8, at most about 6.7, at most about 6.6, at most about 6.5, at most about 6.4, at most about 6.3, at most about 6.2, at most about 6.1, at most about 6.0, at most about 5.5, at most about 5.0, or less than about 5.0. A pH of a cell generated by the systems or methods described herein can be from about 6.5 to about 7.2. A pH of a cell generated by the systems or methods described herein can be from about 6.5 to about 6.55, about 6.5 to about 6.6, about 6.5 to about 6.65, about 6.5 to about 6.7, about 6.5 to about 6.75, about 6.5 to about 6.8, about 6.5 to about 6.85, about 6.5 to about 6.9, about 6.5 to about 6.95, about 6.5 to about 7, about 6.5 to about 7.2, about 6.55 to about 6.6, about 6.55 to about 6.65, about 6.55 to about 6.7, about 6.55 to about 6.75, about 6.55 to about 6.8, about 6.55 to about 6.85, about 6.55 to about 6.9, about 6.55 to about 6.95, about 6.55 to about 7, about 6.55 to about 7.2, about 6.6 to about 6.65, about 6.6 to about 6.7, about 6.6 to about 6.75, about 6.6 to about 6.8, about 6.6 to about 6.85, about 6.6 to about 6.9, about 6.6 to about 6.95, about 6.6 to about 7, about 6.6 to about 7.2, about 6.65 to about 6.7, about 6.65 to about 6.75, about 6.65 to about 6.8, about 6.65 to about 6.85, about 6.65 to about 6.9, about 6.65 to about 6.95, about 6.65 to about 7, about 6.65 to about 7.2, about 6.7 to about 6.75, about 6.7 to about 6.8, about 6.7 to about 6.85, about 6.7 to about 6.9, about 6.7 to about 6.95, about 6.7 to about 7, about 6.7 to about 7.2, about 6.75 to about 6.8, about 6.75 to about 6.85, about 6.75 to about 6.9, about 6.75 to about 6.95, about 6.75 to about 7, about 6.75 to about 7.2, about 6.8 to about 6.85, about 6.8 to about 6.9, about 6.8 to about 6.95, about 6.8 to about 7, about 6.8 to about 7.2, about 6.85 to about 6.9, about 6.85 to about 6.95, about 6.85 to about 7, about 6.85 to about 7.2, about 6.9 to about 6.95, about 6.9 to about 7, about 6.9 to about 7.2, about 6.95 to about 7, about 6.95 to about 7.2, or about 7 to about 7.2.

Monitoring can generate data relating to the characteristics of the cells. Monitoring can generate data relating to the progress of processes that are currently being performed, have been performed, or are planned to be performed in a system. Data can generated and collected using data analysis pipelines, such as IP data analysis pipeline 136, 336 and QC data analysis pipeline 138, 338. The data can be generated via the monitoring and can be used to generate an output report. The output report can be assessed by a human operator to track the progress of the system. An output report can be a progress report 170, 370. For example, a progress report can indicate that the cell has been selected but has not been subjected to an activation reaction. For example, a progress report can indicate that the cell have been selected but have not been subjected to an activation reaction. A user can use the report to monitor the system without interfering with the processes. Characterization of the cells at a final stage can allow for the quality control of the therapeutic product. A release report, such as release report 180, 380 can be generated based on the characterization and potency of the cells.

The system can be a closed system and can be operated in a sterile environment. The minimization of contamination throughout the process can allow for a higher yield of cells that are viable and effective for cell therapy. The system can comprise container or consumable for the storage and manipulation of cells. The consumables can be sterile consumables. The consumables can be single use. Single use sterile consumables can allow for a sterile manufacturing environment and decrease contamination of the cells at any stage in the process.

The system can be a fully automated system. The system can require minimal to no user input during the processes. The fully automated system can allow a user to initiate the system and without any addition input, the system can provide a therapeutic cell. This can remove human error from the manufacturing process and allow for saving of costs and time. Where the system does require sample output or input, utilization of sterile connections and transfer containers is supported to maintain the closed system.

### Computer Control Systems

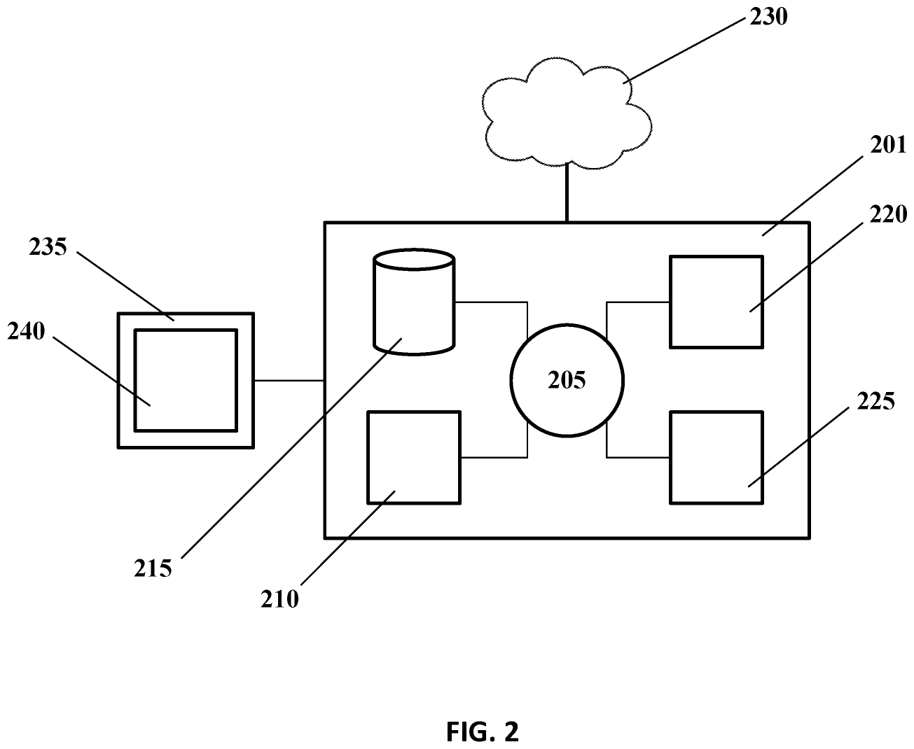

The present disclosure provides computer control systems that are programmed to implement methods of the disclosure. FIG. 2 shows a computer system 201. that is programmed or otherwise configured to perform processes described throughout this disclosure. The computer system 201 can regulate various aspects of systems or modules of the present disclosure, such as, for example, receive data from modules, initiate the processing of cells in a module, manipulate cells, monitor reactions, or provide reports The computer system 201 can be an electronic device of a user or a computer system. that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which can enable devices coupled to the computer system 201 to behave as a client or a server.

The CPU 205 can execute a sequence of machine-readable instructions, which can be embodied in a program or software. The instructions can be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system. 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., laboratory technician). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine- executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 201 can include or be in communication with an electronic display 235 that comprises a user interface (UI) 240 for providing, for example, status reports of the system, data relating to the characteristic of the cells. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, process sequencing data generated to identify the presence of a particular gene of interest in a cell.

### EXAMPLES

### Example 1. Generation of a Therapeutic Cells

A subject can be suffering from a disorder and can benefit from treatment with cell therapy. A sample can be taken from a subject and collected in a blood bag or apheresis bag. FIG. 1 shows a schematic of system for generating cells for therapy. A blood bag 101 comprising cells of a subject is hooked up a system. The cells are moved into module 110 which performed initial reactions on the cells. The module 110 can receive the sample via sample input and assessment 112 which can receive samples of whole blood or apheresis products. The module can provide a fast early assessment of the cell count and viability along with a immunophenotyping of the starting material to provide information on the starting material for later processes. After sample input and assessment, the module 110 performs a cell selection 114 to select for a set of cells, for example, naïve T-cells, for modification and activation. Cell activation 116 is then performed by introducing a stimuli to the cells (e.g., T-cells) to generate activated cells. Cell activation 116 can be performed by introducing beads comprising antibodies that interact with the cells act cause activation. Cell transduction 118 is then performed by adding in nucleic acids and a nucleic acids vector to integration of nucleic acids. Cells transfection of other forms of the nucleic acid manipulation can also be performed using a mechanical method or non-viral only method. 118 can include introduction of exogenous genes into the cells, for example, chimeric antigen receptors (CARs) and can generate CAR-T cells. The newly transduced and activated cells are then moved to module 120 to allow for cell culture expansion and proliferate the cells. Module 130 is configured to perform monitoring of the process of module 110 and 120. Module 130 can be directly integrated with module 110 and 120 or can be a standalone module. Module 130 performs in-line monitoring and characterization 132 and characterizes cells for in other modules. For example, the phenotype of cells can be characterized. Module 130 can also perform NOS library preparation 134 by extracting nucleic acids from the cells and the subjecting these nucleic acids to library preparation and NGS sequencing 160. The expanded cells subjected to third party analysis 150 to determine the quality of these expanded therapeutic cells. Module 130 uses the data from monitoring, sequencing, and third part analysis to an inputted into an in line processing data analysis pipeline 136 and a Quality Control Data analysis pipeline 138. The pipelines 136 and 138 generates reports such as a progress report 170 relating to the status of the systems and cells, and a release report 180 relating to the quality metrics of the final expanded cells. Upon expansion of cells, the cells are moved to module 140 for harvesting 142, formulation and filling 144, and cryopreservation 146. The cells are harvested and then aliquoted in a formulation and the preserved to longer storage. The cells are ready for use by a subject such as therapeutically ready cells 190, such as CAR-T cells. The cells can then be provided to a subject for treatment.

FIG. 3 shows a schematic of an example system for generating therapeutic cells. In FIG. 3, the QC Module 130 is expanded to show components of In-Process Monitoring and Characterization 332 and Release QC and Characterization. The Release QC and Characterization has the process the NGS library preparation 334. Testing aliquots can be taken directly from the bioreactor, frozen samples, or in-process development and are introduced into the QC Module 130. These sample inputs are aliquoted out into the format used within the QC Module 130 that utilizes multi-omics data from sequencing and computer vision analysis to be inputted into an off-line processing data analysis pipeline 136, 336 and a Quality Control Data analysis pipeline 138, 338. The analysis pipelines are executed in parallel and leverage some information from intermediate steps that generate reports such as a progress report 170, 370 relating to the status of the systems and cells and a release report 180, 380 relating to the quality metrics of the final expanded cells. Each assay is provided from the analysis as a single result or is combined with other assays to deliver a multi-omics interpretation of the cells and product. Some modules, like the Cell Count and Viability module, are utilized within the QC device, bioreactor, or as a single module or instrument. The Quality Control Data analysis pipeline has multiple components including, but not limited to, vector copy number (VCN) identification, a replication competent lentivirus (RCL) identification, a chimeric antigen receptor (CAR) expression level, an immunophenotyping, or a combination thereof.

Provided below are exemplary embodiments of the invention:

Embodiment 1: A system for generating therapeutic cells, wherein the system comprises: (a) a first module configured to receive a plurality of cells from a subject, wherein the first module is further configured to (i) select a subset of cells from the plurality of cells to yield selected cells; and (ii) introduce nucleic acids into the selected cells to yield modified cells; (b) a second module configured to culture the modified cells to generate expanded cells; and (c) a third module configured to harvest the expanded cells, thereby generating therapeutic cells, wherein the third module is further configured to (i) generate a formulation comprising the therapeutic cells; and (ii) preserve the therapeutic cells, wherein the system is a closed, automated system.

Embodiment 2: The system of embodiment 1, wherein the first module is further configured to activate the selected cells to generate activated cells.

Embodiment 3: The system of embodiment 1 or 2, wherein the first module is configured to introduce nucleic acids into the selected cells by transduction to generate transduced cells.

Embodiment 4: The system of embodiment 1 or 2, wherein the first module is configured to introduce nucleic acids into the selected cells by transfection.

Embodiment 5: The system of embodiment 3, wherein the system is further configured to identify at least one characteristic of the plurality of cells, the selected cells, the activated cells, the transduced cells, or the expanded cells.

Embodiment 6: The system of any one of embodiments 1-5, further comprising a fourth module configured to monitor the first module or the second module.

Embodiment 7: The system of embodiment 6, wherein the fourth module is further configured to identify a characteristic of the plurality of cells, the selected cells, the activated cells, the transduced cells, or the expanded cells.

Embodiment 8: The system of any one of embodiments 1-7, wherein the system is further configured to perform a quality analysis on the expanded cells to determine a quality of expanded cells.

Embodiment 9: The system of embodiment 8, wherein the quality analysis comprises forming a nucleic acid library, optionally wherein the nucleic acid library comprises a next generation sequencing library.

Embodiment 10: The system of embodiment 9, wherein the quality analysis comprises identifying a vector copy number (VCN), a replication competent lentivirus (RCL), a chimeric antigen receptor (CAR) expression level, an immunophenotyping, or a combination thereof.

Embodiment 11: The system of any one of embodiments 5-10, wherein the at least one characteristic comprises a phenotype of a cell of the plurality of cells, the selected cells, the activated cells, the transduced cells, or the expanded cells.

Embodiment 12: The system of embodiment 11, wherein the phenotype of the cell is a T cell, a B cell, or a NK cell.

Embodiment 13: The system of any one of embodiments 5-12, wherein the at least one characteristic comprises a genotype of a cell of the plurality of cells, the selected cells, the activated cells, the transduced cells, or the expanded cells.

Embodiment 14: The system of any one of embodiments 5-13, wherein the at least one characteristic comprises a viability of a cell of the plurality of cells, the selected cells, the activated cells, the transduced cells, or the expanded cells.

Embodiment 15: The system of any one of embodiments 5-14, wherein the at least one characteristic comprises a presence of a contaminant.

Embodiment 16: The system of embodiment 15, wherein the contaminant is a bacterial contaminant, a mold contaminant, a viral contaminant, a yeast contaminant, or a mycoplasma contaminant. Embodiment 17: The system of embodiment 15, wherein the contaminant is an endotoxin.

Embodiment 18: The system of any one of embodiments 5-17, wherein the at least one characteristic comprises an image of a cell of the plurality of cells, the selected cells, the activated cells, the transduced cells, or the expanded cells.

Embodiment 19: The system of any one of embodiments 5-18, wherein the at least one characteristic comprises a potency of a cell of the plurality of cells, the selected cells, the activated cells, the transduced cells, or the expanded cells.

Embodiment 20: The system of any one of embodiments 5-19, wherein the at least one characteristic comprises a pH, a temperature, an oxygen level, or a metabolic composition of a cell of the plurality of cells, the selected cells, the activated cells, the transduced cells, or the expanded cells.

Embodiment 21: The system of any one of embodiments 1-20, wherein the first module is further configured to monitor the selected cells or modified cells.

Embodiment 22: The system of any one of embodiments 1-21, wherein the second module is further configured to monitor the expanded cells.

Embodiment 23: The system of any one of embodiments 1-22, wherein the plurality of cells are in a blood bag or apheresis bag.

Embodiment 24: The system of any one of embodiments 1-22, wherein the plurality of cells are from a biological sample.

Embodiment 25: The system of any one of embodiments 1-24, wherein the plurality of cells comprise immune cells.

Embodiment 26: The system of any one of embodiments 1-25, wherein the third module is further configured to cryopreserve the therapeutic cells.

Embodiment 27: The system of any one of embodiments 1-26, wherein the therapeutic cells are chimeric antigen receptor (CAR) T cells.

Embodiment 28: A method for generating therapeutic cells, wherein the method comprises: (a) receiving a plurality of cells from a subject; (b) selecting a subset of cells from the plurality of cells to yield selected cells; (c) introducing nucleic acids into the selected cells to yield a modified cell; (d) culturing the modified cell to generate expanded cells; (e) harvesting the expanded cells, thereby generating therapeutic cells; (f) generating a formulation comprising the therapeutic cells; and (g) preserving the therapeutic cells, wherein the method is performed in a closed automated system.

Embodiment 29: The method of embodiment 28, further comprising prior to c) and subsequent to b), activating a cell from the selected cells to yield an activated cell, wherein c) comprises introducing nucleic acids into the activated cells.

Embodiment 30: The method of embodiment 28 or 29, wherein the introducing nucleic acids into the selected cells comprises transfection.

Embodiment 31: The method of embodiment 28 or 29, wherein the introducing nucleic acids into the selected cells comprises transduction to generate transduced cells.

Embodiment 32: The method of embodiment 31, wherein the transduction comprises using a viral vector.

Embodiment 33: The method of embodiment 32, wherein the viral vector is a lentiviral vector.

Embodiment 34: The method of any one of embodiments 31-33, further comprising identifying at least one characteristic of the plurality of cells, the selected cells, the activated cells, the transduced cells, or the expanded cells.

Embodiment 35: The method of any one of embodiments 28-34, further comprising performing quality analysis on the expanded cells to determine quality of expanded cells.

Embodiment 36: The method of embodiment 35, wherein the quality analysis comprises forming a nucleic acid library, optionally wherein the nucleic acid library comprises a next generation sequencing library.

Embodiment 37: The method of embodiment 36, wherein the quality analysis comprises identifying a vector copy number (VCN), a replication competent lentivirus (RCL), a chimeric antigen receptor (CAR) expression level, an immunophenotyping, or a combination thereof.

Embodiment 38: The method of any one of embodiments 34-37, wherein the at least one characteristic comprises a phenotype of a cell of the plurality of cells, the selected cells, the activated cells, the transduced cells, or the expanded cells.

Embodiment 39: The method of embodiment 38, wherein the phenotype of the cell is a T cell, a B cell, or a NK cell.

Embodiment 40: The method of any one of embodiments 34, wherein the at least one characteristic comprises a genotype of a cell of the plurality of cells, the selected cells, the activated cells, the transduced cells, or the expanded cells.

Embodiment 41: The method of any one of embodiments 34-40, wherein the at least one characteristic comprises a viability of a cell of the plurality of cells, the selected cell, the activated cell, the transduced cell, or the expanded cells.

Embodiment 42: The method of any one of embodiments 34-41, wherein the at least one characteristic comprises a presence of a contaminant.

Embodiment 43: The method of embodiment 42, wherein the contaminant is a bacterial contaminant, a mold contaminant, a viral contaminant, a yeast contaminant, or a mycoplasma contaminant.

Embodiment 44: The method of embodiment 42, wherein the contaminant is an endotoxin.

Embodiment 45: The method of any one of embodiments 34-44, wherein the characteristic comprises an image a cell of the plurality of cells, the selected cell, the activated cell, the transduced cell, or the expanded cells.

Embodiment 46: The method of any one of embodiments 28-45, wherein the plurality of cells are in a blood bag or apheresis bag.

Embodiment 47: The method of any one of embodiments 28-45, wherein the plurality of cells are from a biological sample.

Embodiment 48: The method of any one of embodiments 28-47, wherein the plurality of cells comprise immune cells.

Embodiment 49: The method of any one of embodiments 28-48, wherein the preserving comprises cryopreserving the therapeutic cells.

Embodiment 50: The method of any one of embodiments 28-49, wherein generating a formulation comprises adding a cryoprotectant to the therapeutic cells.

Embodiment 51: A non-transitory computer readable medium comprising instructions that, when executed by a computer processor, cause the computer processor to automatically control a closed system to (a) receive a plurality of cells from a subject; (b) select a subset of cells from the plurality of cells to yield selected cells; (c) introducing nucleic acids into the selected cells to yield modified cells; (d) culture the modified cells to generate expanded cells; (e) harvest the expanded cells, thereby generating therapeutic cells; (f) generate a formulation comprising the therapeutic cells; and (g) preserve the therapeutic cells.

Embodiment 52: A non-transitory computer readable medium comprising instructions that, when executed by a computer processor, cause the computer processor to automatically control a closed system to perform the method of any of embodiments 28-49.

Embodiment 53: A non-transitory computer readable medium comprising instructions that, when executed by a computer processor, cause the computer processor to automatically control the system of any of embodiments 1-27.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein can be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A system for generating therapeutic cells, wherein the system comprises:
(a) a first module configured to receive a plurality of cells from a subject, wherein the first module is further configured to
(i) select a subset of cells from the plurality of cells to yield selected cells; and
(ii) introduce nucleic acids into the selected cells to yield modified cells;
(b) a second module configured to culture the modified cells to generate expanded cells; and
(c) a third module configured to harvest the expanded cells, thereby generating therapeutic cells, wherein the third module is further configured to
(i) generate a formulation comprising the therapeutic cells, and
(ii) preserve the therapeutic cells;
wherein the system is a closed, automated system.

2. The system of claim 1, wherein the first module is further configured to activate the selected cells to generate activated cells.

3. The system of claim 1, wherein the first module is configured to introduce nucleic acids into the selected cells by transduction to generate transduced cells.

4. The system of claim 1, wherein the first module is configured to introduce nucleic acids into the selected cells by transfection.

5. The system of claim 3, wherein the system is further configured to identify at least one characteristic of the plurality of cells, the selected cells, the activated cells, the transduced cells, or the expanded cells.

6. The system of claim 1, further comprising a fourth module configured to monitor the first module or the second module.

7. The system of claim 6, wherein the fourth module is further configured to identify a characteristic of the plurality of cells, the selected cells, the activated cells, the transduced cells, or the expanded cells.

8. The system of claim 1, wherein the system is further configured to perform a quality analysis on the expanded cells to determine a quality of expanded cells.

9. The system of claim 8, wherein the quality analysis comprises forming a nucleic acid library, optionally wherein the nucleic acid library comprises a next generation sequencing library.

10. The system of claim 9, wherein the quality analysis comprises identifying a vector copy number (VCN), a replication competent lentivirus (RCL), a chimeric antigen receptor (CAR) expression level, an immunophenotyping, or a combination thereof.

11. The system of claim 5, wherein the at least one characteristic comprises a phenotype of a cell of the plurality of cells, the selected cells, the activated cells, the transduced cells, or the expanded cells.

12. The system of claim 11, wherein the phenotype of the cell is a T cell, a B cell, or a NK cell.

13. The system of claim 5, wherein the at least one characteristic comprises a genotype of a cell of the plurality of cells, the selected cells, the activated cells, the transduced cells, or the expanded cells.

14. The system of claim 5, wherein the at least one characteristic comprises a viability of a cell of the plurality of cells, the selected cells, the activated cells, the transduced cells, or the expanded cells.

15. The system of claim 5, wherein the at least one characteristic comprises a presence of a contaminant.

16. The system of claim 15, wherein the contaminant is a bacterial contaminant, a mold contaminant, a viral contaminant, a yeast contaminant, or a mycoplasma contaminant.

17. The system of claim 15, wherein the contaminant is an endotoxin.

18. The system of claim 5, wherein the at least one characteristic comprises an image of a cell of the plurality of cells, the selected cells, the activated cells, the transduced cells, or the expanded cells.

19. The system of claim 5, wherein the at least one characteristic comprises a potency of a cell of the plurality of cells, the selected cells, the activated cells, the transduced cells, or the expanded cells.

20. The system of claim 5, wherein the at least one characteristic comprises a pH, a temperature, an oxygen level, or a metabolic composition of a cell of the plurality of cells, the selected cells, the activated cells, the transduced cells, or the expanded cells.

21. The system of claim 1, wherein the first module is further configured to monitor the selected cells or modified cells.

22. The system of claim 1, wherein the second module is further configured to monitor the expanded cells.

23. The system of claim 1, wherein the plurality of cells are in a blood bag or apheresis bag.

24. The system of claim 1, wherein the plurality of cells are from a biological sample.

25. The system of claim 1, wherein the plurality of cells comprise immune cells.

26. The system of claim 1, wherein the third module is further configured to cryopreserve the therapeutic cells.

27. The system of claim 1, wherein the therapeutic cells are chimeric antigen receptor (CAR) T cells.

28. A method for generating therapeutic cells, wherein the method comprises:
(a) receiving a plurality of cells from a subject;
(b) selecting a subset of cells from the plurality of cells to yield selected cells;
(c) introducing nucleic acids into the selected cells to yield a modified cell;
(d) culturing the modified cell to generate expanded cells;
(e) harvesting the expanded cells, thereby generating therapeutic cells;
(f) generating a formulation comprising the therapeutic cells; and
(g) preserving the therapeutic cells,
wherein the method is performed in a closed automated system.

29. The method of claim 28, further comprising prior to c) and subsequent to b), activating a cell from the selected cells to yield an activated cell, wherein c) comprises introducing nucleic acids into the activated cells.

30. The method of claim 28, wherein the introducing nucleic acids into the selected cells comprises transfection.

31. The method of claim 28, wherein the introducing nucleic acids into the selected cells comprises transduction to generate transduced cells.

32. The method of claim 31, wherein the transduction comprises using a viral vector.

33. The method of claim 32, wherein the viral vector is a lentiviral vector.

34. The method of claim 31, further comprising identifying at least one characteristic of the plurality of cells, the selected cells, the activated cells, the transduced cells, or the expanded cells.

35. The method of claim 28, further comprising performing quality analysis on the expanded cells to determine quality of expanded cells.

36. The method of claim 35, wherein the quality analysis comprises forming a nucleic acid library, optionally wherein the nucleic acid library comprises a next generation sequencing library.

37. The method of claim 36, wherein the quality analysis comprises identifying a vector copy number (VCN), a replication competent lentivirus (RCL), a chimeric antigen receptor (CAR) expression level, an immunophenotyping, or a combination thereof.

38. The method of claim 34, wherein the at least one characteristic comprises a phenotype of a cell of the plurality of cells, the selected cells, the activated cells, the transduced cells, or the expanded cells.

39. The method of claim 38, wherein the phenotype of the cell is a T cell, a B cell, or a NK cell.

40. The method of claim 34, wherein the at least one characteristic comprises a genotype of a cell of the plurality of cells, the selected cells, the activated cells, the transduced cells, or the expanded cells.

41. The method of claim 34, wherein the at least one characteristic comprises a viability of a cell of the plurality of cells, the selected cell, the activated cell, the transduced cell, or the expanded cells.

42. The method of claim 34, wherein the at least one characteristic comprises a presence of a contaminant

43. The method of claim 42, wherein the contaminant is a bacterial contaminant, a mold contaminant, a viral contaminant, a yeast contaminant, or a mycoplasma contaminant.

44. The method of claim 42, wherein the contaminant is an endotoxin.

45. The method of claim 34, wherein the at least one characteristic comprises an image a cell of the plurality of cells, the selected cell, the activated cell, the transduced cell, or the expanded cells.

46. The method of claim 28, wherein the plurality of cells are in a blood bag or apheresis bag.

47. The method of claim 28, wherein the plurality of cells are from a biological sample.

48. The method of claim 28, wherein the plurality of cells comprise immune cells.

49. The method of claim 28, wherein the preserving comprises cryopreserving the therapeutic cells.

50. The method of claim 28, wherein generating a formulation comprises adding a cryoprotectant to the therapeutic cells.

51. A non-transitory computer readable medium comprising instructions that, when executed by a computer processor, cause the computer processor to automatically control a closed system to
(a) receive a plurality of cells from a subject;
(b) select a subset of cells from the plurality of cells to yield selected cells;
(c) introducing nucleic acids into the selected cells to yield modified cells;
(d) culture the modified cells to generate expanded cells;
(e) harvest the expanded cells, thereby generating therapeutic cells;
(f) generate a formulation comprising the therapeutic cells; and
(g) preserve the therapeutic cells.

52. A non-transitory computer readable medium comprising instructions that, when executed by a computer processor, cause the computer processor to automatically control a closed system to perform the method of any of claims 28-50.

53. A non-transitory computer readable medium comprising instructions that, when executed by a computer processor, cause the computer processor to automatically control the system of any of claims 1-27.
